(19) United States
(12) Patent Application Publication
(10) Pub. No.: US 2004/0167430 A1
(43) Pub. Date: Aug. 26, 2004

(54) Cutaneous biopsy device with handle and disposable tips

(76) Inventors: Babak B. Roshdieh, Fresno, CA (US); Martin E. Smith, Camarillo, CA (US)

Correspondence Address: KENNETH J. HOVET, NORDMAN, CORMANY, HAIR & COMPTON, P.O. BOX 9100, 1000 TOWN CENTER DRIVE, OXNARD, CA, 93031-9100, US

(21) Appl. No.: 10/617,232

(22) Filed: Jul. 9, 2003

Related U.S. Application Data
(63) Continuation-in-part of application No. 10/371,277, filed on Feb. 20, 2003.

(51) Int. Cl. A61B 10/00

(52) U.S. Cl. 600/567

(57) ABSTRACT

A skin punch device comprises a gripping handle with a center passage, through which a reciprocating plunger extends. A disposable cutting tip is releasably connected to the handle. The cutting tip has a tip cavity with a base from which extends a biopsy chamber that terminates at cutting edges. The base has an aperture through which a slidable push-rod extends. The push-rod has a disc end located in the biopsy chamber. The plunger and push-rod are in axial alignment, so that when the plunger is moved, the push-rod will also move in tandem. Movement of the push-rod will simultaneously cause the disc end to pass through the biopsy chamber and eject a cut tissue sample lodged in the chamber. Further movement of the plunger will result in engagement with the tip cavity and dislodgement of the entire disposable cutting tip from the end of the gripping handle.

This Application is a Continuation-In-Part of application Ser. No. 10/371,277, filed Feb. 20, 2003, entitled CUTANEOUS BIOPSY DEVICE WITH HANDLE AND DISPOSABLE TIPS.

### BACKGROUND OF THE INVENTION

1. Field of the Invention

The present invention relates to the field of biopsy tools. More specifically, the invention relates to a biopsy cutting tool comprising a re-usable handle and a disposable tip for obtaining cutaneous tissue samples.

1. Description of Related Art

Advances in modern medicine rely increasingly on advanced diagnostic procedures to identify pathogens and diseases at an early stage. One of the more important diagnostic techniques is the securing of a tissue sample for biopsy. A cutaneous biopsy is a skin sample taken for a variety of purposes, including diagnosing the presence of melanoma, carcinoma, and other forms of skin cancer.

A tool for taking a cutaneous tissue sample comprises a cutting edge formed along an outer edge of a cylindrical barrel. The cutting edge is pressed against tissue and the barrel is rotated, cutting into the tissue and obtaining a cylindrically shaped tissue sample.

In order to retrieve the tissue sample from the cylindrical barrel, in the past, physicians or lab technicians have been known to skewer the tissue sample with a needle and pry it out of the barrel shaped blade. In light of the possibility of cutting oneself on the contaminated cutting blade while trying to retrieve the tissue sample, it can be readily appreciated that such a procedure has potentially grave health risks to the lab technician or physician.

U.S. Pat. No. 3,692,020 to Schied teaches a gun-shaped housing within which is positioned a hollow spindle to which is attached a ratchet mechanism actuated by a trigger. An ejector rod is biased outwardly by a spring which extends through the spindle. At the rod's inner end is located a disk, which is positioned within the upper end of the cutting die. In operation, the spindle is wound-up by the knob against the bias torque spring. Upon actuation of the trigger, the spring will cause a reverse unwinding which will rotate the cutter to induce a cutting action against the tissue. Upon completion of the cutting action, the die is lifted form the skin and the ejector rod is depressed. The depression of the ejector rod forces the disk against tissue sample, thereby ejecting the specimen from the die interior. Schied, however, does not teach a disposable blade and tip, and re-sterilization must be performed after each use.

U.S. Pat. Nos. 5,183,053 and 5,186,178 to Yeh et al. discloses a disposable handle in which a disposable blade is pressed fitted. The entire unit is then discarded after use. Yea et al. do not teach a reusable handle. Additionally, Yeh et al. do not teach a means for ejecting the tissue safely from the elliptically shaped blade after securing the biopsy sample, and the lab technician is forced to remove the tissue sample by some makeshift means.

U.S. Pat. No. 5,833,628 to Yuan et al. teaches a disposable bone graft harvester having a clear, graduated plastic tube tipped with a bone cutting head at one end and a handle or other torque supplying connection at the other end. An ejector rod having an enlarged disc-like end is fitted within a tissue-receiving sleeve. At the end of the sleeve is a cutting head comprising rotating blades which remove bone samples which are then accumulated in the sleeve. The cutting head is subsequently removed, and the ejector rod is depressed to deposit the cut tissue into a selected receptacle.

Yuan et al. is not directed to a cutaneous biopsy device, but rather, to a bone graft harvester. Instead of securing a single contiguous tissue sample, Yuan et al has rotating blades that scrape away small flecks of bone with each rotation. Moreover, Yuan et al. does not teach a re-usable handle which does not need to be subjected to re-sterilization after each use.

U.S. Pat. No. 6,155,989 to Collins discloses a syringe-like disposable cutaneous biopsy instrument equipped with a tubular blade at its lower end, and designed so that a vacuum is created during use. The vacuum serves to retain undeformed plug of tissue cut from a patient's skin. Collins does not teach a reusable member. He also does not teach a second plunger that is separated from contaminated fluids and tissue.

U.S. Pat. No. 6,440,086 to Hohenberg describes an internal biopsy device comprising a needle for piercing tissue and attaining a desired depth for an internal biopsy. Hohenberg is not directed to a cutaneous biopsy device, and he does not teach a cutting head which is removable by actuating a plunger.

### SUMMARY OF THE INVENTION

The biopsy device of the present invention overcomes the aforementioned prior art disadvantages by providing a cutting means that is readily removed from a handle means and is disposable. The cutting means further includes a discharging means for ejecting tissue samples into a storage receptacle. A coupling means is also provided to releasably attach the cutting means to the handle means. Additionally, the handle means includes a release means that advantageously allows rapid release of the cutting means from the handling means.

More particularly, the device comprises a handle to which is attached a plunger and a disposable cutting tip member. The handle comprises an elongated structure having a longitudinal center passage that extends from an open upper end to a lower discharge end. The plunger is in sliding engagement with inner surface portions of the center passage and includes an inner plunger head that is spaced above the free end of a push rod that moves axially within an aperture in the tip member. The tip member includes a base from which extend tip sidewalls that define an open tip cavity. Extending from the base in a direction opposite from the tip sidewalls, are cutter walls. The cutter walls terminate at sharp cutting edges which are used to cut into a patient's skin. The cutter walls define a biopsy chamber for temporarily housing a skin biopsy sample prior to being ejected into a storage receptacle.

The base is provided with a center aperture through which extends the push rod. The push rod comprises a shaft with a free end which extends into the tip cavity. At the shaft's opposing end is an enlarged disc having a periphery that corresponds to the cross-sectional shape of the biopsy chamber.

The tip cavity sidewalls releasably engage the handle discharge end, and includes an abutment means for engaging the plunger contact end (head) to cause dislodgement of the tip member when the plunger is moved to a fully depressed position. Prior to engagement with the abutment means, the plunger will be in an intermediate position whereby the plunger contact end will engage the push rod free end and cause the opposing disc end to move axially into the biopsy chamber. The opposing disc end functions to displace any biopsy contents that may be in the chamber as a result of tissue cutting during operation of the device.

The plunger has an actuating end that projects outwardly beyond the handle's open upper end to permit manual depression of the plunger. Axial alignment of the plunger is provided by its sliding engagement with an interior reduced diameter section of the center passage. The center passage includes a compression spring to bias the plunger outwardly in a non-operative stowable position.

The handle discharge end is provided with tip attachment means for releasably engaging the tip member. It comprises an outwardly extending annular connecting structure with connector walls having an outer engagement surface that may be tapered. The cross-sectional shape of the connecting structure corresponds to the cross-sectional shape of the tip cavity sidewalls. This arrangement permits the formation of a friction engagement joint between the tip cavity inner surfaces and the connecting structure outer engagement surfaces. Alternative tip engagement means are offsetting annular beads, or an annular bead on one surface with a corresponding annular groove on the opposing surface, or axially aligned ribs, serrations and dimples on one or both of the joint surfaces. An elastic O-ring or other resilient friction engagement structure could also be utilized between the joint surfaces to effect a releasable connection.

The present invention further comprehends the use of a tray means to display an array of cutting tip members having a variety of cutting edge shapes and sizes. The tip members are oriented in the tray means with respective tip cavities uppermost. In this orientation, a user can dislodge and discard a used tip member and subsequently engage a selected fresh tip member. This is conveniently accomplished by positioning the handle tip attachment means over the tip cavity of the selected tip member and pushing the handle down until the joint surfaces are effectively engaged. The tray means may include compartments for holding different sizes and types of tip members. The entire tray means will be sterilized and overlaid with a sealed covering. Alternatively, each compartment can be overlaid with an individual cover to avoid contamination of adjacent compartments.

The invention also provides a method of operating a cutaneous tissue-sampling device having a cutting edge formed on the edge of a substantially cylindrical member. The method comprises the steps of pressing a cutting edge of the cutaneous tissue-sampling device against a tissue, wherein a handle of the cutaneous tissue-sampling device is oriented on a conceptual plane at a reference angle of zero degrees, rotating the cutaneous tissue-sampling device around an axis of the reference angle while maintaining pressure against the tissue; depressing the cutting edge into the tissue, thereby cutting a tissue sample within the substantially cylindrical member; angling the handle of the cutaneous tissue-sampling device to a positive angle of orientation on the conceptual plane; rotating the cutaneous tissue-sampling device around an axis of the positive angle of orientation, thereby severing the tissue sample from an underlying connective tissue; and withdrawing the cutaneous tissue-sampling device. The above actions will permit the tissue to remain lodged within the substantially cylindrical member of the tissue sampling device. As such, a significant problem in the prior art has been overcome wherein prior attempts to lift the sample from surrounding tissue by forceps, toothpicks and the like, caused damage to the tissue.

At least the biopsy chamber of the tip member may be transparent. This feature helps a user in making an accurate biopsy cut. Also, it allows the use of gradations which indicate a depth of tissue cut by the blade.

The cutting edges may define a curved or polygonal opening. The cutting edge may be constructed of a moldable plastic or resin material to produce a cutting edge of surgical quality without the use of machine tooling or abrasion. Not excluded, however, is the use of metal alone, or in combination with plastics or resins, to construct various parts of the biopsy device.

### BRIEF DESCRIPTION OF THE DRAWINGS

FIG. 1 is an isometric view of a cutaneous biopsy device comprising a reusable handle and a disposable tip.

FIG. 2 is a cross-sectional view taken along lines 2-2 of FIG. 1.

FIG. 3 is a fragmentary cross-sectional view of the front portion of the FIG. 1 device depicting dislodgement of a biopsy specimen.

FIG. 4 is a view similar to FIG. 3 depicting the beginning of disengagement of the disposable tip.

FIG. 5 is a view similar to FIG. 3 illustrating complete disengagement of the disposable tip for the handle.

FIG. 6 is a top plan view of a package containing disposable tips.

FIG. 7 is an enlarged cross-sectional view taken along line 7-7 of FIG. 6.

FIG. 8 is an enlarged cross-sectional view of a transparent disposable tip.

FIG. 9 is an enlarged fragmentary cross-sectional view of the tip separated from the handle wherein frictional engagement occurs with a bead and groove joint.

FIG. 10 is a fragmentary cross-sectional view of a first embodiment of a biopsy chamber constructed of metal cutter walls embedded into the tip member base.

FIG. 11 is a fragmentary cross-section view of a second embodiment of metal cutter walls with a base flange fastened to the tip member base.

FIG. 12 is a fragmentary cross-sectional view of a third embodiment of metal cutter walls having a cap structure engaged to the tip member base and sidewalls.

FIG. 13 is a fragmentary cross-sectional view of a fourth embodiment of thin metal cutter walls embedded into the tip member base and having serrated cutting edges.

FIG. 14 is a fragmentary cross-sectional view of a fifth embodiment of metal cutter walls forming an overlap joint with a plastic tip member base element.

FIG. 15 is a fragmentary cross-sectional view of a sixth embodiment of metal cutter walls forming a lap joint with a plastic tip member base element.

FIG. 16 is a fragmentary cross-sectional view of a 7^{th} embodiment of metal cutter walls forming a threaded joint with a plastic tip member base element.

### DESCRIPTION OF THE PREFERRED EMBODIMENTS

The present invention is directed to a cutaneous biopsy device comprising a disposable tip 30 coupled to a re-usable handle 10. The handle includes a plunger 27 and the tip includes a push rod 33 that interacts with the plunger during operation of the device.

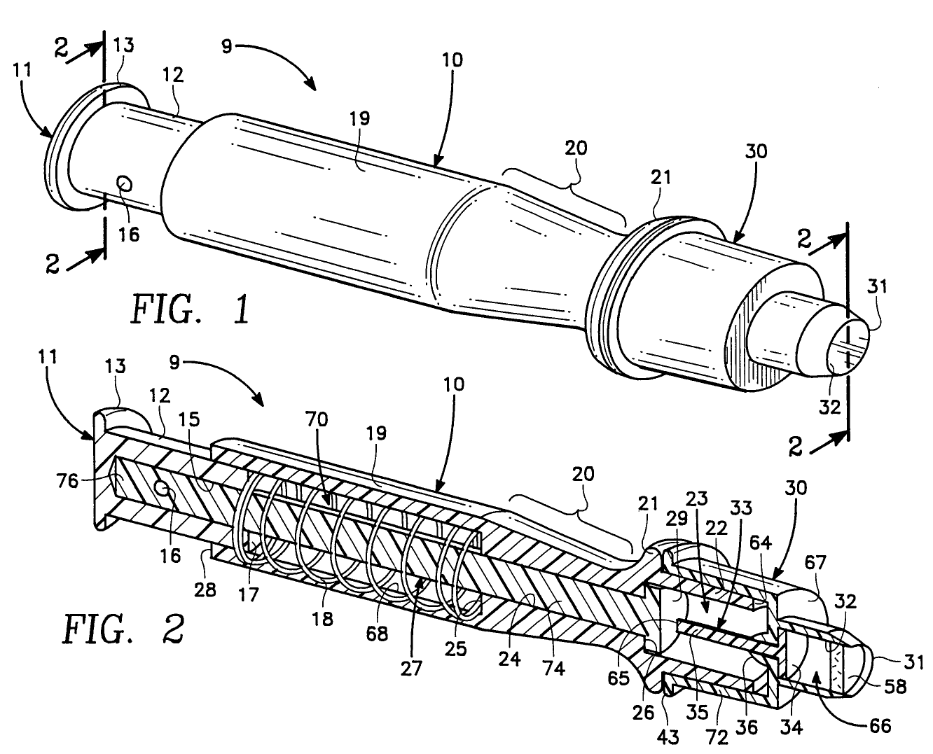

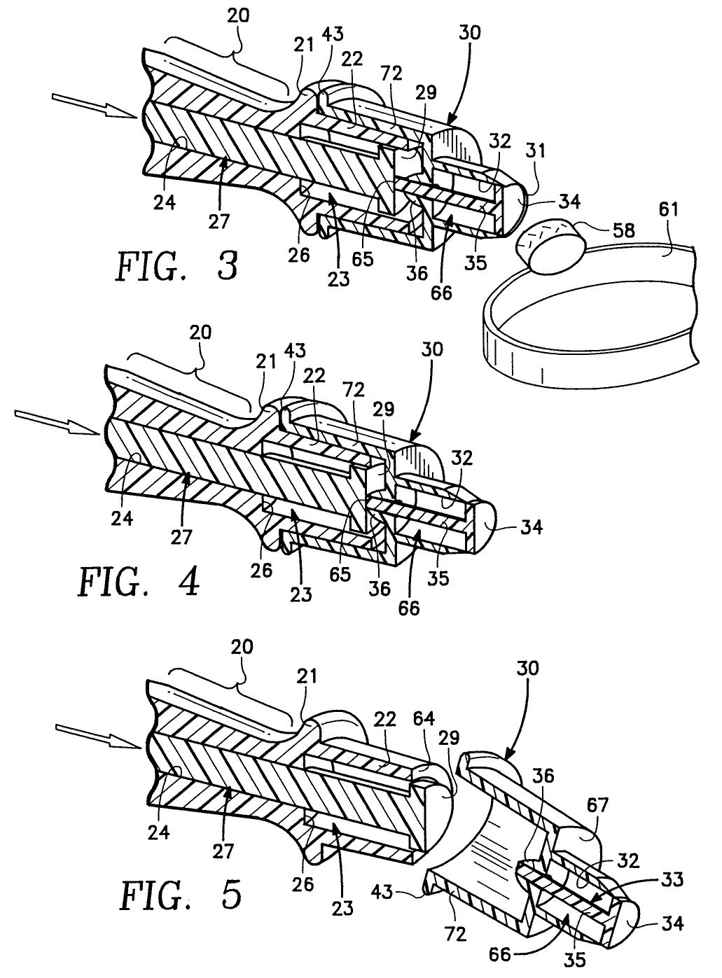

FIG. 1 illustrates a fully assembled biopsy device. FIG. 2 is a cross-sectional view of the biopsy device of FIG. 1 illustrating the component parts fitted together and interacting as a unit. FIGS. 3-5 illustrate operation of the device.

With reference now to FIGS. 1 and 2, handle 10 includes a grip region 19. Formed within the grip region is a longitudinal center passage 24. The passage extends from an open upper end 28 to a lower discharge end 64. The plunger 27 is in sliding engagement with inner surface portions of the center passage 70, and includes an inner contact head 29 that is spaced above free end 65 of push-rod 33 that moves axially within the cutting tip member 30. The tip member comprises a base 67 from which extend tip sidewalls 72 that define an open tip cavity 23.

Extending from base 67 in a direction opposite from the tip sidewalls, are cutter walls 32. The cutter walls terminate at sharp edges 31, which are used to cut into a patient's skin. The cutter walls define a biopsy chamber 66 for temporarily housing a skin biopsy specimen 58 upon completion of the cutting process.

The center passage includes an enlarged portion 68 extending from handle open end 28 to inner ledge 25. The longitudinal extent of the large portion corresponds generally to the length of grip region 19. The upper channel ledge 25 creates the beginning of a reduced diameter section 24 of the central passage. This section further extends toward the lower handle discharge end 64, until it merges into a radially outwardly extending shoulder 26. The connecting wall outer surfaces may optionally be tapered to provide a frictional engagement with the inner surfaces of the tip cavity sidewalls 72.

The reusable plunger 27 slidably fits within the center passage 70. It comprises a plunger shaft 74 having a lower end that merges into plunger head 29. The plunger upper end is fastened to a button 11. The plunger shaft has a diameter that is slightly less than the reduced diameter section 24 of the center passage. The plunger head 29 has a disc-like shape with a diameter less than the interior diameter of handle connector walls 22. The plunger has sufficient length to position the plunger head 29 within tip cavity 23 while also extending to an attachment end 76 beyond handle open end 28.

Button 11 is a structure having an actuating head 13 from which extends a body portion 12. The body portion terminates at inner edge 17. Extending axially into the inner edge is a button bore 15. Attachment end 76 of the plunger shaft extends into the button bore and is fastened to body portion 12 with securing pin 16 in a manner known in the art.

A compression spring 18 is disposed within the center passage enlarged portion 68. The spring has a lower end that rests against enlarged passage ledge 25, and an upper end that abuts against button inner edge 17. When in a first stowable position as shown in FIG. 2, the spring will be uncompressed and the button actuating head 13 will be spaced above handle open end 28. As the actuating head is depressed, the spring 18 will exert a counter force against button inner edge 17 and shoulder 25, thereby resisting depression of the button. The button 11 and plunger 27 thereby advance and retract as a single unit, as described below.

As the spring 18 forces button 11 upward to the first position, the reusable plunger also moves upward until plunger head 29 engages shoulder 26 formed at the junction of connector wall 22 and the reduced diameter section 24. This engagement prevents further retraction of the plunger. It also prevents the button from moving beyond handle open end 28. This arrangement further ensures that, when pressure is not being exerted against the button, the plunger head 29 will not inadvertently advance to exert pressure on push rod 33, nor on boss 36 of the disposable tip 30. As discussed below, this is important in that it ensures that a tissue sample will not be prematurely ejected from the biopsy chamber, and that the tip member 30 will not be prematurely separated from the handle.

The handle 10 is ergonomically designed to fit the human hand, and includes a grip region 19 that merges into a tapered section 20. The tapered section terminates at finger interface flange 21. The tapered section 20 and the finger interface flange 21 afford a user various ways to grip the cutaneous biopsy device during use, including placing the index finger and middle finger below the interface flange 21 like a syringe and activating the button by the force of one's thumb, or by wrapping one's fingers around the grip region, little finger adjacent to the interface flange21, and thumb pointing up and positioned to depress button 11.

Preferably, the tip member 30 comprises a tip cavity 23 and biopsy chamber 66 that are cylindrical. The tip cavity is defined by a base 67 from which extend tip sidewalls 72. The tip sidewalls extend upwardly to an outwardly flared lip 43. As best shown in FIGS. 1 and 2, when the tip member is fully engaged with handle connector walls 22, the lip 43 will abut against the underside of interface flange 21.

With reference to FIG. 8, extending through the base 67, coextensive with the cavity center axis, is a rod aperture 37. Preferably, the base includes an inwardly extending boss 36 through which the rod aperture also extends. The boss adds length to the rod aperture for greater lateral strength. It also provides an abutment means for engagement with plunger head 29 during operation of the device.

Extending outwardly from below base 67 is a cutter wall 32. The cutter wall is coaxial with the device longitudinal center axis and terminates at cutting edge 31.

A disposable push rod 33 comprising an enlarged disc part 34 and a rod shaft 35, is oriented within the tip member such that the disc part is in biopsy chamber 66, and the rod shaft passes through rod aperture 37 and into tip cavity 23.

The diameter of the rod aperture is preferably almost the same diameter as rod shaft 35. The relative diameters permit the rod shaft to move through the rod aperture in a smooth snug alignment with the device center axis. The snug fit also prevents bodily fluids from leaking through the rod aperture and contaminating reusable parts of the device. Additionally, having a snug fit will inhibit the push rod from sliding entirely out of the rod aperture during disengagement of the biopsy specimen 58.

Under magnification, virtually all cutting edges are seen to be less than perfectly sharp. At a certain point of magnification, cutting edges typically comprise jagged and rough edges. The level of magnification necessary to reveal these imperfections is a measure of the quality of a cutting edge. A cutaneous biopsy device, such as the present invention, utilizes a round cutting edge that is pressed into skin, rotated and swivelled to sever the skin tissue at a selected depth. To detach the specimen from underlying tissue, the blade must also be tilted. Those skilled in the art will understand that, much like a paper cut, any cutting edge designed to be moved across tissue, need not be as sharp as a cutting edge that must cut tissue by pressure only. Thus, use of a moldable plastic or resin material satisfies industry requirements for biopsy cutting blades.

Moreover, providing a tip member made of plastic allows for the use of a clear plastic material such as polycarbonate and acrylic polymers. This feature of the invention allows a physician or medical practitioner to more easily see the progress of cutting a tissue sample. In particular, it permits a user to better undercut a tissue specimen at a selected depth. FIG. 8 illustrates transparent tip member 38 with gradation lines 59 across the outer surface of cutter wall 32. As a user penetrates a patient's skin with cutting edge 31, the depth of penetration can be determined by observing the upward movement of tissue into the biopsy chamber in relation to the predetermined gradation lines.

FIG. 3 illustrates the discharge of a tissue sample 58 from biopsy chamber 66. Following completion of the tissue cutting step, the plunger 27 and push rod 33 will be in a first position as shown in FIG. 2. Thereafter, the biopsy chamber containing the cut tissue sample 58, will be positioned over tissue receptacle 61 for receiving and storing tissue samples. Subsequently, button 11 is pressed a first distance to a second intermediate position as shown in FIG. 3. The first distance is no less than the axial length of the biopsy chamber. This movement will engage plunger head 29 with push rod free end 65. Continued movement will advance disc part 34 to the outermost open end of the biopsy chamber, defined by cutting edge 31. During the above movements, the disc part will engage the cut tissue sample and push it out of the biopsy chamber, thereby allowing the sample to drop into the aforementioned receptacle 61.

Initially, it will be appreciated that the overall length of the push rod 33 will be no less than the axial length of the biopsy chamber. This is necessary to completely expel the tissue sample from the biopsy chamber during movement through the aforementioned first distance to the second intermediate position as shown in FIG. 4.

Further movement of button 11 from the second position will cause plunger head 29 to engage base 67 of the tip cavity 23. Optionally, boss 36 will be contacted as shown in FIG. 4. Continued axial movement by button 11 will release the friction joint between the outer surface of handle connector wall 22 and the inner surfaces of tip cavity sidewalls 72. Upon reaching the third position shown in FIG. 5, plunger head 29 will be about coextensive with lower handle open end 64. At this position, the tip member is dislodged and the button and plunger will have moved axially a second distance that is no greater than the axial extent of the handle connector walls 22.

Depending on the frictional engagement means being employed, it is likely the tip member will become loose and removable from the handle when the second distance has moved less than half the axial extent of connector walls 22.

FIGS. 1-5 illustrate cavity walls 72 that are flared outwardly for engagement with corresponding inwardly tapered connector walls 22. The wall surfaces are uniform and smooth whereby a forced friction engagement joint is created proximate the area adjacent flange 21. As such, only a short second distance movement will be required to disengage the tip member.

FIG. 9 illustrates an alternative tip member 30′ having a friction joint created by engagement of an annular rib 80 that extends inwardly from the inner surfaces of cavity walls 72′. A corresponding annular groove 82 is formed in the outer surfaces of connector walls 22′. In this embodiment, a snap fit is formed and released upon a predetermined axial movement of plunger 27. The location of the above joint structures along the axial extent of the connector wall and cavity wall, will dictate the aforementioned second distance.

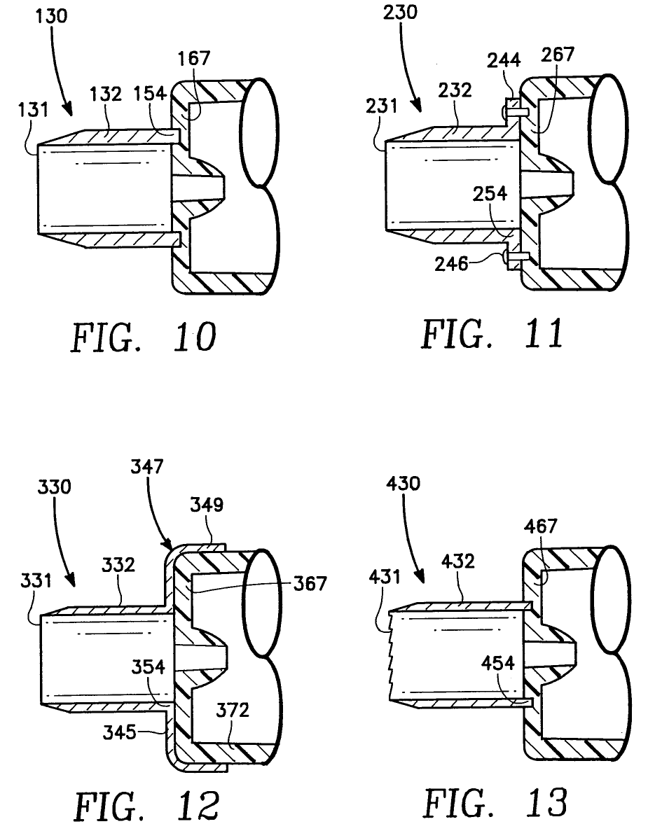

In addition to the above embodiments, it will be appreciated that the entire tip member 30 shown in FIGS. 1-5, and the tip member 30′ shown in FIG. 9, could be constructed entirely of metal. Moreover, because a significant feature of the present invention resides in the provision of a disposable tip, greater flexibility occurs in the construction of the tip. For example, the cutting edge, the entire cutter walls, or a portion thereof, characterized as a cutting edge part, may be constructed of a metal. Surgical steels, stainless steels and related metals may be cast or formed by metallurgical processes into a tip member, or selected portions thereof that include a cutting edge. It has been found that such materials can be economically incorporated into the tip to produce a disposable hybrid tip member. This can be done in a variety of ways, and with multiple types of joint means, as depicted without limitation, in FIGS. 10-16.

In FIG. 10, tip member 130 is shown as a first embodiment. This tip member has a biopsy chamber defined by metal cutter walls 132. The walls are relatively thick with a beveled cutting edge 131 constructed in a manner similar to the tip member 30 shown in FIGS. 1-5. The cutter walls terminate at a bottom end 154, which is secured to plastic tip member base 167, as a result of being embedded therein—preferably during a molding process.

FIG. 11 illustrates a second embodiment tip member 230. This tip member comprises metal cutter walls 232, with a beveled cutting edge 231 similar to the biopsy chamber structure shown in FIG. 10. However, the bottom end 254 of the cutter walls is provided with an outwardly flared flange 244. The flange may extend continuously about the cutter wall bottom end 254, or it may be segmented into spaced-apart multiple discrete flanges. The cutter walls 232 are secured to tip member base 267 with fastener means shown as pegs 246. Other fastener means, such as adhesives, chemical and fusion bonding, screws, rivets, nuts, bolts, keys and pins could be used.

A third tip member embodiment 330 is shown in FIG. 12. This embodiment is similar to tip member 30, wherein cutting edge 331 merges into cutter walls 332 which extend to bottom end 354. However, the bottom end is provided with a cap structure shown generally by reference 347. The cap structure includes a radially extending planar ring portion 345 which overlies at least tip member base 367. It may further merge into a downwardly extending annular skirt 349. The ring portion and skirt encompass base 367, and a lower portion of tip side walls 372. The cap structure 347 may fit tightly over the base and side walls to provide an effective frictional engagement. Alternatively, the fastener means set forth above could be used.

FIG. 13 depicts a fourth embodiment 430 of the tip member. This embodiment provides a thin cylindrical metal blade construction referenced as cutter walls 432. The blade cutting edge 431 is illustrated as being serrated to provide an alternative tissue cutting means as distinguished from the even blade edge shown previously. Cutter wall bottom 454 is embedded into tip base 467 for securement in the same manner as described in relation to bottom end 154 of FIG. 10.

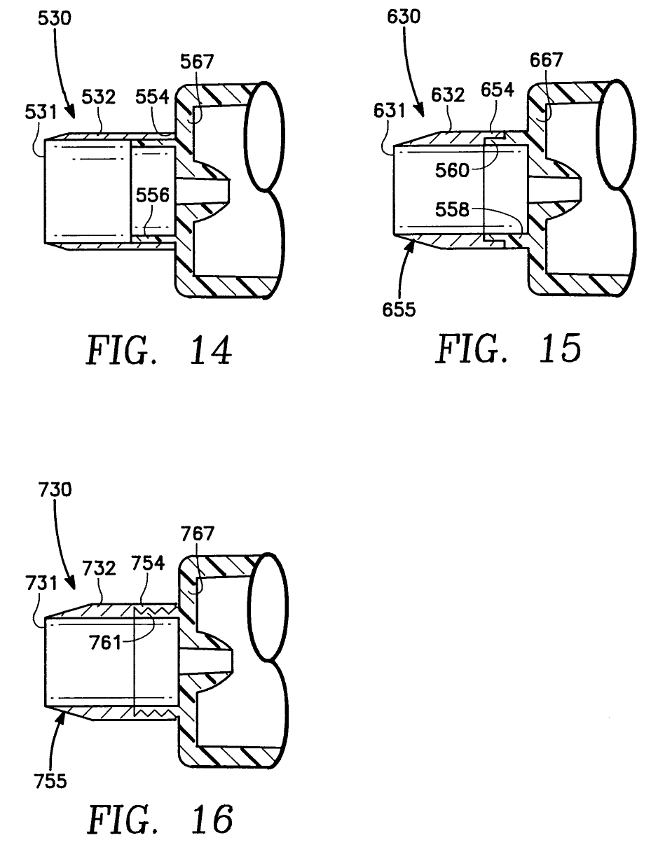

In FIG. 14, a fifth embodiment 530 of the tip member is shown wherein the cutter walls 532 again comprise a thin cylindrical blade-like structure. This structure is defined by the aforementioned cutter walls 532 having a bottom end 554. The outer end of the cutter walls comprise a cutting edge 531, which is shown as an even circular edge. The cylindrical cutter walls are sized to frictionally engage and form an overlap joint with a corresponding base element shown as annular flange member 556. The flange member extends outwardly from base 567 a distance that is sufficient to provide an effective frictional engagement with the corresponding inside surfaces of the cutter walls 532.

It is within the scope of the above connection that cross-pins, keys or other of the aforementioned fastening means could be used at selected axial positions to provide the desired securement at desired cutter wall lengths. Also, the cutter walls 532 or flange member 556 could have relative diameters to create a reverse connection wherein the cutter walls extend inside the flange member.

FIGS. 14-16 provide three examples of alternative joint means for securement of a cutting edge part with a corresponding base element. The cutting edge part comprises a metal cutter wall that extends from the cutting edge to a bottom end. In FIG. 14, an overlap or axially adjustable telescoping joint means is depicted. In FIG. 15, the joint means comprises a lap joint, and in FIG. 16 a threaded joint is provided. Many other types of joints could be used, based upon any one or combination of miter, dado, rabbet, mortise and tenon connections.

With reference to FIG. 15, a sixth embodiment 630 is shown. In this embodiment, tip member base 667 is provided with an outwardly extending annular base flange 558, which defines a base element for the above-referenced joint means. The distal end 560 of the base flange has an annular offset notched configuration. The metal cutter walls 632 define a cutting edge part shown generally by reference 655, that extends from cutting edge 631 to bottom end 654. The bottom end has an annular offset notched configuration that is a mirror image of the above-described base flange distal end configuration. When the bottom end 654 and distal end 560 are fitted together as shown in FIG. 15, a lap joint is formed. The lap joint may be secured by friction engagement, adhesives, fusion bonding, or by appropriate other ones of the fastening means previously mentioned.

In the seventh embodiment 730 shown in FIG. 16, tip base 767 is provided with a base element shown as an outwardly extending threaded base part 761. Corresponding metal cutter walls 732 extend from cutting edge 731 to bottom end 754 to define a cutting edge part shown generally by reference 755. The bottom end 754 includes threads that correspond to the threaded base part 761. When rotated together as shown, the cutting edge part and base part form a secure threaded joint.

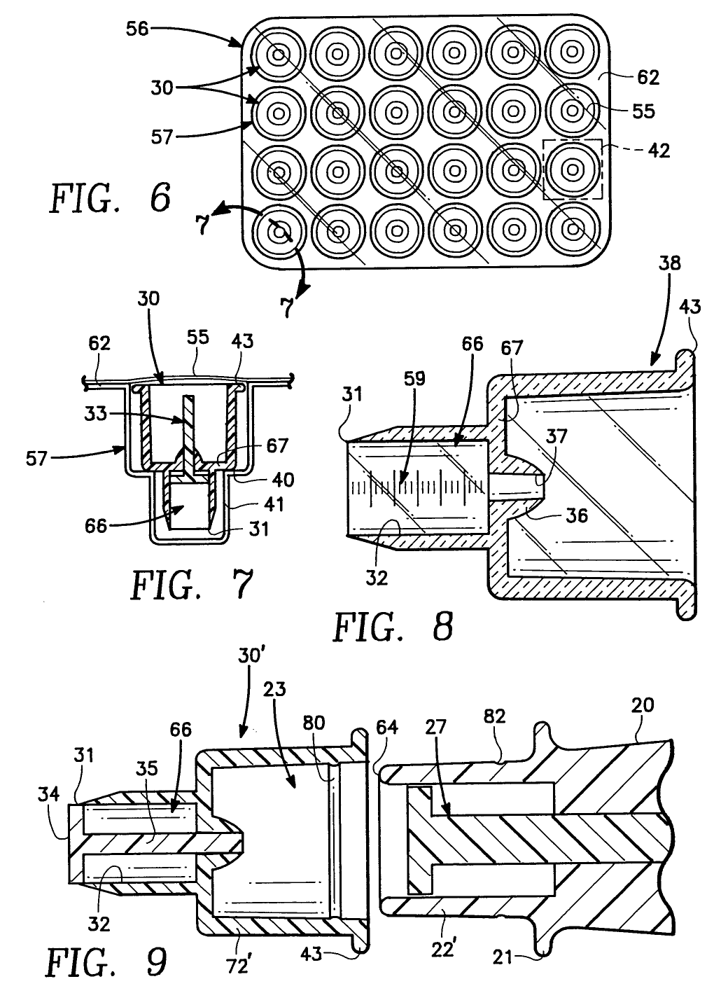

FIGS. 6 and 7 illustrate a package 56 of sterile disposable tip members 30. The package comprises a web 62 from which depend separate compartments 57 for each tip member. The tip members are oriented within each of their respective compartments 57 so that the open tip cavity 23 is uppermost.

It is also desirable to suspend cutting edge 31 above the bottom of the compartment. This is accomplished by providing an inwardly directed annular ledge 40 about the mid portion of compartment 57. The ledge functions to create a reduced diameter well 41 for constraining biopsy chamber 66. The well has a depth greater than the axial extent of the biopsy chamber, so that cutting edge 31 will be spaced above the compartment.

An impermeable cover 55 encloses each of the respective tip members within their respective compartments 57. The cover preferably comprises a heat sealable sheet of plastic, plastic coated foil, cellophane and the like which may be heat sealed or bonded to the web. Because an advantage of the present invention includes the elimination of laboratory sterilization costs by providing a sterile disposable tip to a user, the packaging cover and each tip member is sterilized and sealed to maintain a sealed sterile environment. Individual covers 42, depicted by phantom lines in FIG. 6, may also be used over each compartment. The covering is preferably clear.

According to one embodiment, prior to attaching a sterile tip member within a compartment to handle 10, the cover 55 is peeled back, exposing the sterile disposable tip within the compartment. The handle connector wall 22 is then pushed into tip cavity 23. This action will create the aforementioned friction joint and the tip member can then be lifted directly out of its compartment by movement of the handle without hand contact.

In an alternative embodiment, the handle connector wall may be positioned over cover 55, and forced into the tip cavity without removing the cover. As the handle is withdrawn, the cover will peel away from the package web 55 and become part of the friction joint.

There are multiple advantages to a process wherein the cover is not removed prior to attaching the handle. One is that the user need not waste time picking at a corner of a cover and trying to peel it back. Also, the presence of a thin sheet of cover material between the disposable tip member and the reusable plunger head 29, provides an additional barrier against fluid contamination from the tip member to the handle.

The package, covers and tip members can be sterilized by any known means. According to the preferred embodiment, they are sterilized through radiation sterilization procedures prior to shipping and distribution. The materials comprising the sterile tip and packaging are selected from materials that can withstand sterilization without experiencing undue degradation. Also, the materials used with reusable parts of the device can incorporate bactericide agents known in the art to further guard against contaminants.

Many variations of the foregoing description comprising equivalent structures and processes will be readily apparent to those skilled in the art as falling within the spirit and scope of the claimed invention. Accordingly, the appended claims are to be interpreted according to the widest scope of meaning.

I claim:

1. In a skin biopsy device having a handle connected to a cutting edge, wherein the improvement comprises:
providing said cutting edge as part of a disposable tip member that is releasably connected to said handle, said cutting edge being constructed of metal.

2. The device of claim 1 wherein said disposable tip member is releasably connected to said handle by a friction joint.

3. The device of claim 1 wherein said disposable tip member has a base and includes cutter walls that have a bottom end connected to said base; and,
said cutter walls extending from said bottom end to said cutting edge.

4. The device of claim 3 wherein said bottom end is embedded into said base.

5. The device of claim 3 wherein said bottom end includes an outwardly extending flange that is connected to said base by a fastening means.

6. The device of claim 3 wherein said disposable tip member has side walls and said bottom end includes a cap structure that overlies said base and a portion of said side walls.

7. The device of claim 1 wherein said base has a base element, and said cutter walls comprise a metal cutting edge part connected to said base element with a joint means.

8. A skin biopsy device comprising:
a handle having a center passage and a discharge end;
a plunger positioned within said passage, said plunger having an actuating end and a contact end; and,
a tip member releasably attached to said discharge end, said tip member including a biopsy chamber having an outermost open end defined by a metal cutting edge, said biopsy chamber including a push rod having a disc end in communication with said open end, and a free end in communication with said plunger contact end.

9. The device of claim 8 wherein said plunger is movable from a first position to a second position whereby said plunger contact end abuts against said push rod free end;
said center passage including a biasing means for maintaining said plunger in said first position.

10. The device of claim 8 wherein said tip member has a base and said biopsy chamber is defined by cutter walls extending from said cutting edge to a bottom end which is connected to said base.

11. The device of claim 10 wherein said cutter walls are constructed of metal.

12. The device of claim 11 wherein said cutting edge is serrated.

13. The device of claim 11 wherein said bottom end is embedded in said base.

14. The device of claim 11 wherein said bottom end is connected to said base with a fastening means.

15. The device of claim 11 wherein said bottom end includes a cap structure that overlies at least said base.

16. The device of claim 8 wherein said tip member has a base element and said biopsy chamber includes a cutting edge part, said base element and said cutting edge part being connected by a joint means.

17. The device of claim 16 wherein said cutting edge part is constructed of metal.

18. The device of claim 16 wherein said joint means is any one of a member, or combination of members, selected from a group consisting of lap, overlap, threaded, miter, dado, mortise and tenon, telescope and rabbet.
